(19) United States
(12) Patent Application Publication
(10) Pub. No.: US 2004/0236431 A1
(43) Pub. Date: Nov. 25, 2004

(54) Universal prosthesis

(76) Inventor: Ronald Sekel, Matraville (AU)

Correspondence Address: Thomas M Galgano, Galgano & Burke, 300 Rabro Drive, Suite 135, Hauppauge, NY, 11788, US

(21) Appl. No.: 10/482,602

(22) PCT Filed: Jun. 28, 2002
(86) PCT No.: PCT/AU02/00841

(30) Foreign Application Priority Data
Jun. 28, 2001 (AU) PR 5977

(51) Int. Cl. A61F 2/30; A61B 17/86

(52) U.S. Cl. 623/23.44; 623/23.27; 606/73

(57) ABSTRACT

A universal shaft component capable of insertion as an anchorage in skeletal bone including a proximal humerus, phalange, distal or proximal tibia, distal or proximal femur, or thumb wherein the shaft is insertable axially within an internal bone cavity such that the outer surface of the shaft engages inner walls of said cavity, characterized in that the shaft has a proximal end and a distal end and on said outer surface of said shaft between said ends, at least one thread such that when the shaft is screwed into said bone cavity the at least one thread induces an axial compression force in said bone and distributes that compression force evenly along the bone over the length of the at least one thread.

### BACKGROUND

The present invention relates to surgical prostheses and more particularly relates to a universal component adapted for use in various sites in a skeletal frame. More particularly the invention relates to a shaft component which is adapted for insertion in bone including skeletal joints in the human or animal body and which upon insertion induces a compression in the bone to enhance fixation. More particularly the invention relates to a universal shaft component for insertion in bone and which includes at least one thread whose pitch and angle are constant or vary along the length of the threaded region/s of the shaft. The shaft is particularly applicable to skeletal joints such as but not limited to the shoulder, ankle, thumb, knee and finger where repair or replacement is required. Joint replacements may be required as a result of trauma or disease such as arthritis. Degenerative joints due to such conditions can be extremely disabling and painful often rendering surgery the only treatment for pain relief.

Whilst the present invention will be described principally with reference to its insertion in a selection of joints including the shoulder, knee ankle and finger , it Will be appreciated that the prosthesis component is capable of insertion in other bone sites such as a jaw bone with proportionate reduction or enlargement in size to accommodate the size of the joint the component will replace.

More particularly the present invention provides a universal shaft component for insertion in a bone cavity of human and animal skeletal bone wherein an outer bone engaging surface of the component includes at least one thread having a variable or the same pitch along an axial length of the component which induces and even compression distribution in the bone along the length of the at least one thread.

### PRIOR ART

There are a wide variety of hip prostheses available for repair and replacement of various joints of the human body. Most if not all of these are joint specific and cannot be easily adapted for insertion into other joints of the body due to their purpose built geometry or manner of fixation.

Hip replacements, for example are a common orthopaedic surgical procedure and are usually necessitated by degenerative disease of the hip joint, hip trauma or disease of the hip creating later hip degeneration. There are in existence a number of hip prostheses which have been used to replace the femoral head. Whilst many of the prior art femoral head prostheses have enjoyed widespread use with varying degrees of success, each have suffered from certain attendant disadvantages. The generally known and widely used prostheses typically comprise an arcuate distal shaft having a gradual taper along its full length and terminating proximally in a neck which mates with the head of the prosthesis. The shaft is inserted into the intra medullary cavity of the femur.

This prosthesis is fitted after the surgeon has reamed out the medullary cavity to an extent conducive to the production of tight interfitting between bone and prosthesis when the prosthesis is hammered into position. In practice, the reaming followed by sizing with the prosthesis may be carried out a number of times ie, reaming followed by inserting the prosthesis until there is a small distance of travel of the shaft left near the neck of the femur to enable final hammering into position to thereby create tight interfitting between prosthesis and bone. Femoral explosion both during insertion and extraction is one major drawback when using certain prior art prostheses. However, explosion during insertion is largely due to poor surgical technique or poor prosthesis design which develop high stress forces within bone.

In the past, cementing of the prosthesis has also been employed, however, problems have existed with the use of cement. Failures in hip prostheses have occurred due to loosening at the cement bone interface and at the prosthesis bone interface. In some patients, a rotational failure of the prosthesis can be generated when a patient moves from a seating to a standing position. Also, artificial hips may loosen and fail due to repetitive movement of the distal shaft induced by the locomotion of a wearer. This may eventually lead to a prosthesis failure and possibly unwanted axial dislocation;

One feature of existing hip prostheses is a series of indentations which have been moulded into the distal shaft in order to encourage and stimulate bone growth therein. This bone ingrowth assists in holding the prosthesis firmly in position and also provides a keying and locking effect thereby lessening the possibility of rotational failure and/or unwanted axial subsidence of the prosthesis.

A further problem which exists with this type of prior art prosthesis and in particular with the shaft design is the difficulty in removal from the medullary cavity of a failed prosthesis. A revision hip replacement, necessitates full extraction of the failed prosthesis from the medullary cavity. Where the prosthesis has been held in position by bone growth into the aforesaid recesses of the distal shaft, extraction of the prosthesis can sometimes be extremely difficult, and in some unfortunate instances, may necessitate total longitudinal division of the femur into at least two pieces. Even after division of the femur in this way, a particularly recalcitrant prosthesis firmly affixed to one half of the bone may, in order to effect removal thereof, necessitate further undesirable femoral destruction. Whilst hip prostheses of this type have been in use for some time and have met with considerable field success, the attendant disadvantages of the device are so significant that improvements were necessitated.

Other prosthesis designs are also used having screw threads on the distal shaft however, these suffer from the major disadvantage that it is very difficult for the surgeon to achieve, co-incidence between the correct orientation of the prosthesis at full screw tightness and proper alignment or anteversion between the prosthesis head and the acetabulum. This requires considerable skill on the part of the surgeon with very little margin for error due to the critical alignment and screw tight ness requirements. For this reason surgeons have not utilised the screw prostheses as much as the previously described cemented prosthesis. A further disadvantage of the existing screw prosthesis is its poor resistance to rotational effects which can result in unwanted reverse rotational withdrawal from the femoral medullary cavity. Prior art prostheses employing single screw threads of the same pitch along the length of the shaft have thus been quite unsatisfactory resulting in their limited use. Known hip prosthesis with screw thread is disclosed in the following patents which are incorporated herein by reference; U.S. pat. No. 4,693,724, French patent 2 295729 and European patent 0010527. A hip prosthesis including a spaced apart thread on an axial shaft has been described in international application PCT/AU91/00244 to Sekel which teaches a shaft for insertion and fixation in a femur and in which an axially disposed. compression force is induced in a bone to which it is fixed.

Many types of artificial joints are available for replacement not only in hips but also in joints such as the finger thumb, ankle and major shoulder joint. In shoulder arthroplasty the damaged joint is surgically removed and it is replaced with an artificial joint made of preferably titanium, other metal or both plastic and metal. One prosthesis used in the shoulder replacement is the Neer shoulder system. Whilst the Neer prosthesis has been used successfully in shoulder joint replacement over a number of years there has been no previous use of a shaft which is capable of use in multiple and which induces axial compression in a bone upon and/or during insertion. Also, the prior art does not teach a universal shaft which is insertable in multiple bone sites and joints and which induces a compression in the bone in which the shaft is inserted to enhance fixation. The known bone and particularly joint prostheses have to date been purpose designed for a particular site or more particularly joint in the skeletal frame and are not intended for potential use in multiple sites with the same or substantially the same geometry.

### INVENTION

The present invention is directed to a universal shaft component capable of use in bone and also in skeletal joint replacement in a variety of human (and animal) joints including the hip, shoulder, ankle, finger, knee and thumb. More particularly the invention relates to a universal shaft component capable of use in a plurality of bone sites including the aforesaid joints and including a threaded outer surface wherein the geometry of the thread allows an even compression force to be induced in a bone at the site in which the shaft is inserted. The shaft is preferably adapted to detachably receive a mating component which completes or partially completes the prosthesis for a particular site.

Due to the fundamental form (or geometry) of the shaft, and particularly its absence of curvature which typifies many joint prostheses including hip and shoulder prostheses it is universally applicable to joints and in places in bone where an anchorage post is required such as in a jaw bone.

In its broadest form the present invention comprises:

a universal shaft component capable of fixation in multiple sites in a skeleton; wherein the shaft is insertable axially within an internal bone cavity such that the outer surface of the shaft engages inner walls of said cavity, characterised in that the shaft has a proximal end and distal end; and on said outer surface of said shaft a continuous thread having at least one thread which includes a region of variable pitch such that when the shaft is screwed into said bone cavity the variable pitch of each said at least one thread induces an axial compression force in said bone and distributes that compression force evenly along the bone for the length of the at least one thread. Preferably the at least one thread varies in angle relative to a vertical or horizontal axis along the length of the shaft. Preferably, the shaft includes a first flared end and a second end narrower than the first end, wherein the first end is capable of receiving a mating component.

Preferably the universal shaft is insertable in bone and joints such as but not limited to a hip, shoulder, knee, finger, ankle, thumb.

In another broad form the present invention comprises:

a universal shaft component capable of insertion in a skeletal joint including a hip, shoulder, finger, ankle, thumb wherein the shaft is insertable axially within an internal bone cavity formed in the joint such that the outer surface of the shaft engages inner walls of said cavity, characterised in that the shaft has a proximal end and a distal end and on said outer surface of said shaft between said ends, at least one thread having the same or a variable pitch and angle relative to a horizontal or vertical axis such that when the shaft is screwed into said bone cavity the thread pitch induces an axial compression force in said bone and distributes that compression force evenly along the bone over the length of the thread.

Preferably, the helical thread is continuous along the length of the shaft with a gradual variation from a slow thread at the proximal end to a fast thread towards the distal end. The thread travels in the same direction but due to a regular variation in pitch along the length of the thread from a fast to slow thread an even axial compression distribution is induced in the bone in which the shaft inserted.

An advantage of the gradually varying thread along the shaft is that there is no local compression concentration as the compression forces are distributed evenly.

A universal shaft component capable of fixation in multiple sites in a bone skeleton; wherein the shaft is insertable axially within an internal cavity in bone such that the outer surface of the shaft engages an inner walls of said cavity; wherein the shaft has first and seconds ends wherein one said ends includes a formation which receives and retains a joining component, the outer surface of said shaft including at least one thread having a pitch geometry which when the shaft is screwed into said bone cavity induces an axial compression force in said bone. Preferably, the axial compression force is evenly distributed in the bone along the length of said at least one thread. The Universal shaft includes a flared tapered region with the second end is narrower than the first end. Longitudinal ridges may be provided along the shaft as a key.

According to one embodiment, the shaft has disposed between said first and second ends one helical thread, wherein the pitch of the thread varies as the thread travels axially along the shaft. According to one embodiment , the angle of repose of the thread relative to a vertical or horizontal axis varies along the thread as the thread travels axially along the shaft.

The thread undergoes a variation in pitch from a slow thread near the flared end to a fast thread towards the second end wherein, the thread has a gradual but regular variation in pitch along the length of the thread from a fast to slow thread.

An even compression force is induced in the bone due to differences in travel rates induced on insertion of the shaft by the thread. A profile part at the flared end receives and retains a detachable joining component by male female or female male interfitting . According to one embodiment, the flared end includes a female recess which receives a male end of a joining component.

The shaft is capable of insertion in joints which include glenohumeral joint of the shoulder, a distal end of a femur as a partial knee component, a proximal end of a tibia as a partial knee component, in a proximal phalange to form a part finger joint component, in a distal end of a tibia, in a talus, in a proximal femur.

In another broad form the present invention comprises:

a universal shaft component capable of fixation as an anchorage in multiple sites in a bone skeleton; wherein the shaft is insertable axially within an internal cavity in bone such that the outer surface of the shaft engages an inner wall of said cavity; wherein the shaft has first and seconds ends wherein one said ends includes a formation which receives and retains a joining component, the outer surface of said shaft including first and second spaced apart helical threads; wherein the threads having a pitch geometry which when the shaft is screwed into said bone cavity induces an axial compression force evenly distributed in the bone along the length of said at least one thread. Each thread preferably has a different pitch. Preferably, the angle of repose of a first of the threads relative to a vertical or horizontal axis of the shaft is different from the angle of repose of the second shaft.

According to one embodiment, the first thread is disposed in a region of the shaft near the flared tapered region and the second thread is disposed in a region approximating a longitudinal center of the shaft. The first thread is a slow thread and the second thread is a fast thread wherein the first thread causes a slower axial travel of the shaft than the second thread upon screwing the shaft into a bone cavity. The even compression force is induced in the bone due to differences in travel rates induced on insertion of the shaft by the thread. A profile part at the flared end receives and retains a detachable joining component. The flared end includes a tapered female recess which receives the joining component. One of the threads may have constant pitch or a gradual but regular variation in pitch along the length of the thread from a fast to slow thread.

The double threaded shaft is also capable of insertion in joints which include glenohumeral joint of the shoulder, a distal end of a femur as a partial knee component, a proximal end of a tibia as a partial knee component, in a proximal phalange to form a part finger joint component, in a distal end of a tibia, in a talus, in a proximal femur.

In another broad form the present invention comprises:

A universal shaft component capable of insertion as an anchorage in skeletal bone including a proximal humerus, phalange, distal or proximal tibia, distal or proximal femur, or thumb wherein the shaft is insertable axially within an internal bone cavity such that the outer surface of the shaft engages inner walls of said cavity, characterised in that the shaft has a proximal end and a distal end and on said outer surface of said shaft between said ends, at least one thread such that when the shaft is screwed into said bone cavity the at least one thread induces an axial compression force in said bone and distributes that compression force evenly along the bone over the length of the at least one thread.

In another broad form the present invention comprises:

a universal shaft component for use as an anchorage in a bone and which is capable of forming at least part of a joint replacement in an ankle, hip, finger, thumb, shoulder or knee; the shaft comprising a threaded outer surface which is profiled to induce a compression force in the bone in which it is inserted to enhance fixation and further comprising a flared end and a narrow end, the flared end having a formation which receives and retains a joining member;

### DETAILED DESCRIPTION

The present invention will now be described in more detail according to preferred but non limiting embodiment and with reference to the accompanying illustrations wherein:

FIG. 1 shows a shaft according to one embodiment of the invention with double spaced apart threads.

FIG. 2 shows a shaft according to a preferred embodiment, inserted in a glenohumeral shoulder joint;

FIG. 3 shows the shaft inserted in a distal end of a femur and a proximal end of a tibia to form anchorage for a knee replacement;

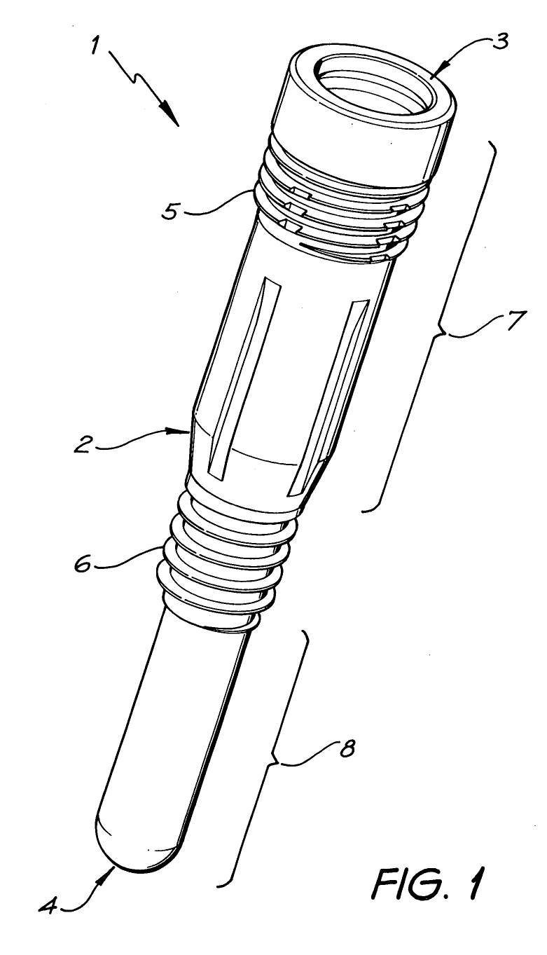

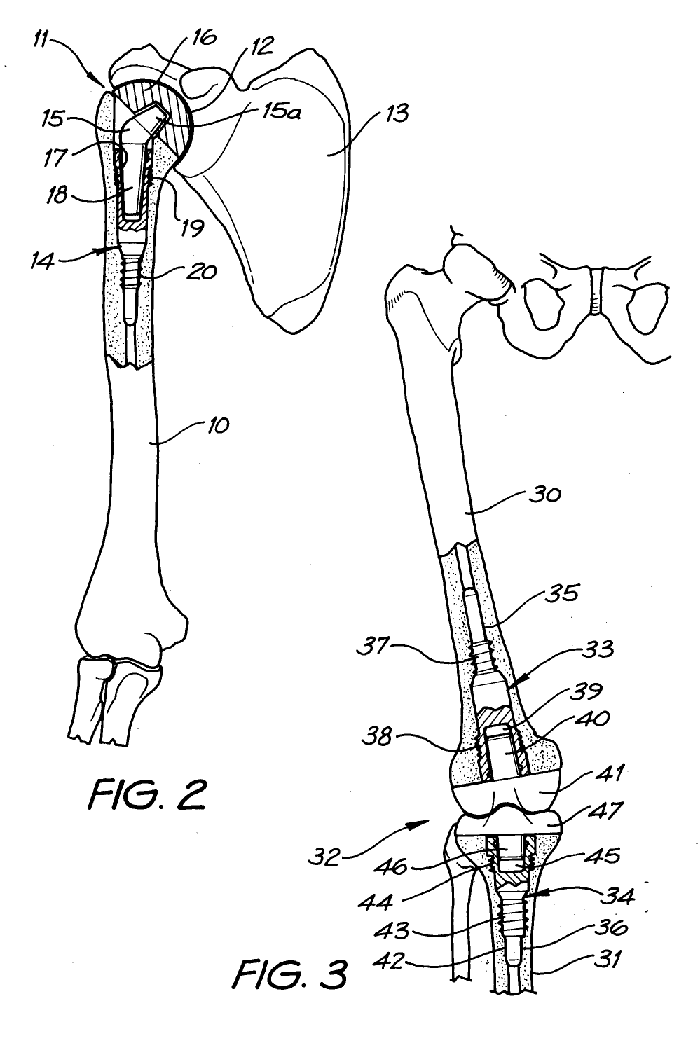

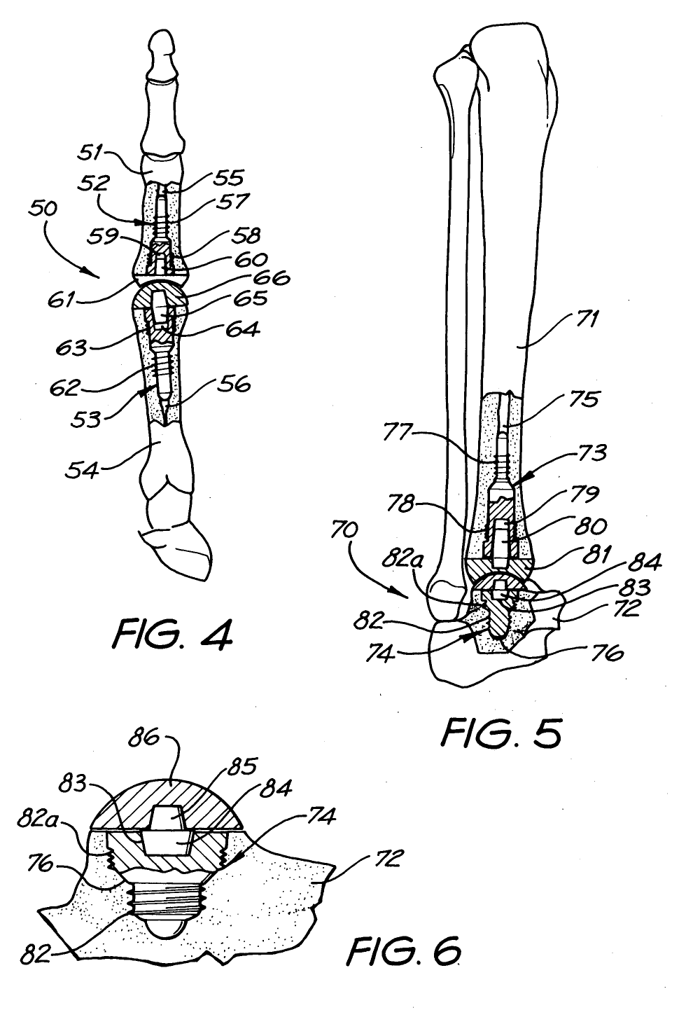

FIG. 4 shows the shaft inserted as a finger joint replacement.

FIG. 5 shows the shaft of FIG. 1 inserted in an ankle joint.

FIG. 6 shows an enlarged view of a shaft inserted in a talus. ankle bone;

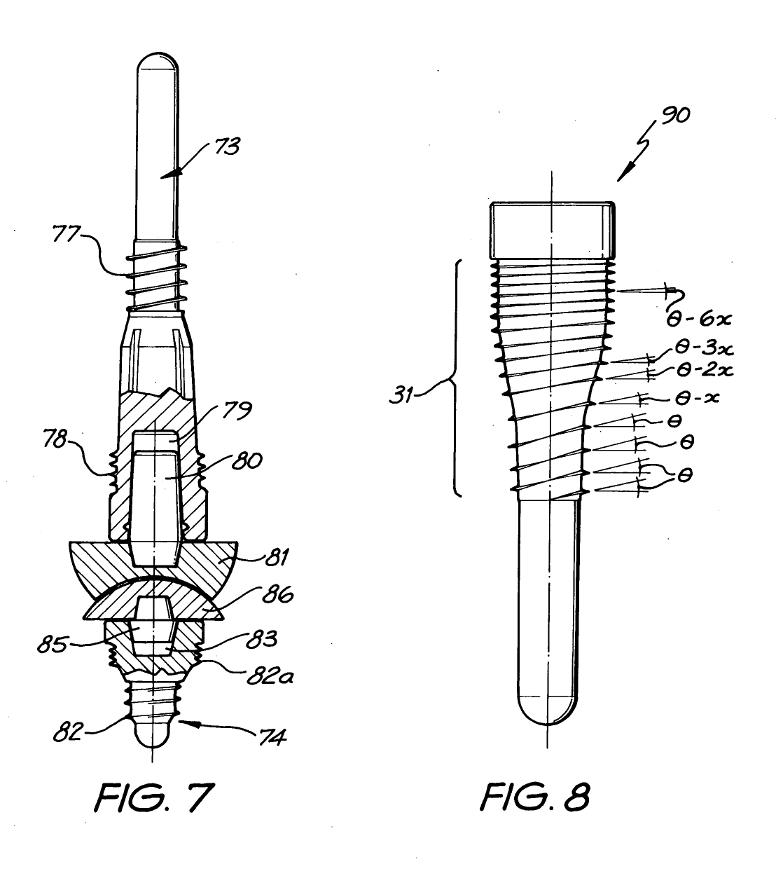

FIG. 7 shows the ankle replacement joint assembly of FIGS. 5 and 6 incorporating two universal shafts.

FIG. 8 shows a universal shaft according to an alternative embodiment with continuous single thread of varying pitch and repose.

FIG. 1 shows a shaft according to a preferred embodiment of the invention.

Universal shaft 1 comprises a shaft body 2 having first and second ends 3 and 4. Intermediate said ends are threads 5 and 6. Threads 5 and 6 are respectively slow and fast threads and due to the difference in axial travel rates induced by the slow and fast threads an even compression is induced in the bone along the length of the thread. First end 3 comprises a flared tapered region 7 and narrow region 8 at second end 4. The prior art teaches the use of prostheses which are purpose built for particular joints. The universal shaft according to the invention has a geometry which enables it to be inserted as a joint component in a wide variety of joints and which is anchored by means of threads which induce a local compression in the bone site upon insertion.

FIG. 2 shows a shaft according to a preferred embodiment, inserted in a glenohumeral shoulder joint;

Referring to FIG. 2 there is shown a simplified view of the glenohumeral joint (right side). This constitutes the major shoulder joint and essentially comprises the humerus 10 which terminates in humerus head 11 which locates in depression 12 in scapularis 13 . The anatomical name of the depression 3 is the glenoid fossa. This joint is held together by extensive muscle and ligament attachments which are not shown. Due to the nature of this joint it is susceptible to arthritis and generally wear over time which can lead to pain in the joint requiring surgical attention. In extreme cases the joint may require replacement. Many surgeons choose to use a Neer prosthesis for replacement of shoulder joints which suffer from osteo arthritis, rheumatoid arthritis, old fractures or fracture dislocations with traumatic arthritis. The shoulder may be totally or partially replaced known as a total or hemi shoulder arthroplasty respectively. There are numerous humeral components used at the present time with choices in respect of head thickness, distal shaft sizes and type, stem length and surface finishes. Prostheses are very often a matter of the surgeon's choice and may also be dictated by the needs of the patient.

FIG. 2 shows a glenohumeral joint replaced with a shaft component 14 according to one embodiment of the present invention. In FIG. 2 the distal shaft is shown connected to an elbow 15 which is in turn connected to a head component 16 which engages the scapular 13. Shaft 14 comprises a recess 17 which receives male taper 18.

In order to insert the prosthesis, the surgeon reams out the cavity of the humerus according to the size of the chosen shaft. Reaming is done approximately to accommodate thread, depth, shaft width and taper. A humerus cavity is reamed to approximately the width of the prosthesis and along the length of a humerus according to the length of the prosthesis shaft taking into account the ultimate alignment between the head of the prosthesis and the glenoid fossa. The Reaming may be done with a tool having a closely made configuration to that of the prosthesis. Shaft 14 is screwed into the medullary cavity if necessary with bone graft supplementation to ensure a strong prosthesis bone bond. Threads 19 and 20 impart an advantage to shaft 14 as they co operate to induce compression that cementing or precoating of the prosthesis is rendered non essential. Nevertheless at the surgeons choice, the prosthesis shaft 14 may be coated with bone growth promotion compounds such as hydroxyapetite. In order to insert the shaft in the humerus 10 the surgeon typically reams out the medullary cavity in order to accommodate shaft 14. Where humerus head 11 is to be replaced this is surgically removed by the surgeon. Shaft 14 is then inserted in the medullary cavity by means of an alien key or with the assistance of a torque wrench. Shaft 14 includes fast thread 14 and slower thread 15 which advance axially at different rates upon rotation of the shaft. This induces a compression in humerus 10 and therefore adequate fixation of shaft 11. As an alternative to spaced apart threads 19 and 20, shaft 11 may be adapted with a single thread along at least part of the length of the shaft (see FIG. 8) having variable pitch thereby inducing an even axial compression in the humerus along the length of the threaded region.

Recess 17 of shaft 14 into which is placed elbow component 15 is tapered so engagement is effected by means of a mutual taper in that recess 17 of shaft 14 is tapered outwardly whereas the mating taper on that end of elbow 15 which engages recess 17 tapered inwardly such that it is narrowest at its extremity. Similarly opposite end 15a of elbow 15 tapers as it extends into the shaft and this engages recess 21 in cup 16. Cup 16 is adapted to move within recess 12 of scapula 13.

The aforesaid describes the present invention with reference to its insertion in a shoulder joint but it will be appreciated that the prosthesis can be universally inserted in other joints in the human body such as but not limited to the ankle, thumb, finger knee and hip.

FIG. 3: shows the shaft inserted in a distal end of a femur and a proximal end of a tibia to form anchorage for a knee replacement;

Referring to FIG. 3 there is shown a simplified view of a knee joint (right side) with opposing shafts. This joint essentially comprises the femur 30 and tibia 31. Due to the nature of this joint it like the shoulder is susceptible to arthritis, injury and generally wear over time which can lead to pain in the joint requiring surgical attention. In extreme cases the joint may require replacement. There are numerous knee components available for use at the present time with choices including size, type, material and surface finishes. The selection will usually be dictated by the needs of the patient.

FIG. 3 shows a knee joint 32 including a shaft component 33 located distally in femur 30. The joint further includes opposing shaft component 34 located in tibia 31. Components 33 and 34 are respectively inserted in cavities 35 and 36 prepared respectively in the in the medullary cavity of the femur 30 and tibia 31.

In order to insert shaft 33 the surgeon reams out the cavity 35 in femur 30 according to the size of the chosen shaft component. Reaming is done approximately to accommodate thread, depth, shaft width and taper so cavity 35 is a close fit to the outer contour of shaft 33. Cavity 35 is reamed to approximately the width of the prosthesis allowing for taper and along the length of femur 30 according to the length of shaft 33 taking into account the ultimate knee alignment required. As with insertion of the shaft in the shoulder and other joints the reaming may be done with a reaming tool having a configuration close to that of the shaft. Shaft 33 is screwed into the medullary cavity 35 if necessary with bone graft supplementation to ensure a strong prosthesis bone bond. Threads 37 and 38 co operate to induce a compression force in the distal region of femur 30. Cementing or precoating of the prosthesis is rendered non essential. Nevertheless at the surgeons choice, the shaft 33 may be coated with bone growth promotion compounds such as hydroxyapetite. Shaft 33 is then inserted in the medullary cavity 35 by means of an alien key or with the assistance of a torque wrench. Threads 37 and 38 advance axially at different rates upon rotation of the shaft, thereby inducing a compression in femur 30 enhancing fixation. As an alternative to spaced apart threads 37 and 38, shaft 33 may be adapted with a single thread along at least part of the length of the shaft (see FIG. 8) having variable pitch thereby inducing an even axial compression in femur 30 along the length of the threaded region.

Shaft 33 includes a female recess 39 into which is placed a male tapered stem 40 of liner 41 which completes the femoral component of knee 32. This engagement is effected by means of a mutual taper in that recess 39 of shaft 33 is tapered outwardly whereas the mating tapered stem 40 which engages recess 39 is tapered inwardly such that it is narrowest at its free end.

Shaft 36 is inserted in tibia 31 in a similar manner to that described for the insertion of shaft 33. Shaft 36 is screwed into the medullary cavity 42. Threads 43 and 44 co operate to induce a compression force in the proximal region of Tibia 31. The shaft 36 may be coated with bone growth promotion compounds such as hydroxyapetite.

Threads 43 and 44 advance axially at different rates upon rotation of shaft 36, thereby inducing a compression in Tibia 31 enhancing fixation. As an alternative to spaced apart threads 43 and 44, shaft 36 may be adapted with a single thread along at least part of the length of the shaft (see FIG. 8) having variable pitch thereby inducing an even axial compression in Tibia 31 along the length of the threaded region.

Shaft 36 includes a female recess 45 into which is placed a male tapered stem 48 of knee platform liner 47 which completes the Tibial component of knee 32. This engagement is effected by means of a mutual taper in that recess 45 of shaft 36 is tapered outwardly whereas the mating tapered stem 48 which engages recess 45 is tapered inwardly such that it is narrowest at its free end.

The tapered connections formed by engagement of stems 40 and 48 with respective recesses 39 and 45 allow for some rotational alignment prior to driving home the stems.

FIG. 4 shows the shaft inserted as a finger joint replacement.

Referring to FIG. 4 there is shown a simplified view of a finger joint 50 with opposing shafts. Due to the nature of this joint it is susceptible to arthritis, injury and generally wear over time which can lead to pain in the joint requiring surgical attention. In extreme cases the joint may require replacement.

FIG. 4 shows a proximal phalange 51 including a shaft component 52 located proximally. The joint further includes opposing shaft component 53 located in bone 54. Components 52 and 53 are respectively inserted in cavities 55 and 56.

In order to insert shaft 52 and 53, the surgeon reams out the cavity 55 and 56 according to the size of the chosen shaft component. Reaming is done approximately to accommodate thread, depth, shaft width and taper so cavities 55 and 56 are a close fit for components 52 and 53. As with insertion of the shaft in the shoulder and other previously described joints the reaming is done with a reaming tool having a configuration close to that of the shaft. Also if required bone graft supplementation may be employed to ensure a strong prosthesis bone bond. As in the previously described joint applications threads 57 and 58 of shaft 52 co operate to induce a compression force in the phalange 51 as a result of thread 57 inducing faster axial travel on rotation of shaft 52 than is induced by slow thread 58. Thus threads 57 and 58 advance axially at different rates upon rotation of the shaft, thereby inducing a compression enhancing fixation.

As an alternative to spaced apart threads 57 and 58, shaft 52 may be adapted with a single thread along at least part of the length of the shaft (see FIG. 8) having variable pitch thereby inducing an even axial compression in phalange 51 along the length of the threaded region.

Shaft 52 includes a female recess 59 into which is placed a male tapered stem 60 of articulating platform 61 which completes the proximal phalange component of finger joint 50. This engagement is effected by means of a mutual taper in that recess 59 of shaft 52 is tapered outwardly whereas the mating tapered stem 60 which engages recess 59 is tapered inwardly such that it is narrowest at its free end.

Shaft 53 is inserted in bone 54 in a similar manner to that described for the insertion of shaft 52. Shaft 53 is screwed into the medullary cavity 56 and threads 62 and 63 co operate to induce a compression force. Threads 62 and 63 advance axially at different rates upon rotation of shaft 53, thereby inducing a compression enhancing fixation. As an alternative to spaced apart threads 62 and 63 of shaft 53 may be adapted with a single thread along at least part of the length of the shaft (see FIG. 8) having variable pitch thereby inducing an even axial compression along the length of the threaded region.

Shaft 53 includes a female recess 64 into which is placed a male tapered stem 65 of articulating platform liner 66 which completes the artificial finger joint 50. This engagement is effected by means of a mutual taper in that recess 64 of shaft 53 is tapered outwardly whereas the mating tapered stem 65 which engages recess 64 is tapered inwardly such that it is narrowest at its free end.

The tapered connections formed by engagement of stems 60 and 65 with respective recesses 59 and 64 allow for some rotational alignment prior to driving home the stems.

FIG. 5 shows the shaft inserted in an ankle joint including a distal tibia 71 and talus 72. Shaft component 73 is located proximally in tibia 71. The joint 70 further includes opposing shaft component 74 which is abbreviated to accommodate the limited space available in talus 72. Components 71 and 74 are respectively inserted in preformed cavities 75 and 76.

In order to insert shaft components 73 and 74, as previously described the surgeon reams out the cavities 75 and 76 according to the size of the chosen shaft component. Reaming is done approximately to accommodate thread, depth, shaft width and taper so cavities 75 and 76 provide a close fit for components 73 and 74. As with insertion of the shaft in the shoulder and other previously described joints the reaming is done with a reaming tool having a configuration close to that of the shaft. Also if required bone graft supplementation may be employed to ensure a strong prosthesis bone bond. Threads 77 and 78 of shaft 73 co operate to induce a compression force in tibia 71 as a result of thread 77 inducing faster axial travel on rotation of shaft 73 than is induced by slow thread 78. Thus threads 77 and 78 advance axially at different rates upon rotation of the shaft, thereby inducing a compression enhancing fixation in tibia 71.

As an alternative to spaced apart threads 77 and 78, shaft 73 may be adapted with a single thread along at least part of the length of the shaft (see FIG. 8), having variable pitch thereby inducing an even axial compression in tibia 71 along the length of the threaded region.

Shaft 73 includes a female recess 79 into which is placed a male tapered stem 80 of articulating platform 81 which completes the proximal tibial component of ankle joint 70. This engagement is effected by means of a mutual taper in that recess 79 of shaft 73 is tapered outwardly whereas the mating tapered stem 80 which engages recess 79 is tapered inwardly such that it is narrowest at its free end.

FIG. 6 shows an enlarged view of a shaft 74 inserted in a talus ankle bone 72. Shaft 74 is inserted in talus 72 in a similar manner to that described for the insertion of shaft 73. Shaft 74 is screwed into cavity 76 and threads 82a and 82 co operate to induce a compression force in talus 72 enhancing fixation of shaft 74.

Shaft 74 includes a female recess 83 into which is placed a male tapered stem 84. Stem 84 has at its opposite end a male taper 85 which receives and retains thereon articulating platform 86 which completes the talus component of joint 70. The engagement is effected by means of a mutual taper in that recess 83 of shaft 74 is tapered outwardly whereas the mating tapered stem 65 which engages recess 64 is tapered inwardly such that it is narrowest at its free end.

The tapered connections formed by engagement of stems 80 and 84 (see FIG. 6) with respective recesses 79 and 83 allow for some rotational alignment prior to driving home the stems.

FIG. 7 shows the ankle replacement joint assembly of FIGS. 5 and 6 incorporating two universal shafts detached from ankle joint 70. FIG. 7 has corresponding numbering as for the components described in FIGS. 5 and 6.

FIG. 8 shows a universal shaft 90 according to an alternative embodiment with continuous single thread of varying pitch and repose. Threads with the steepest angle are preferably parallel.

In each of the above examples of insertion of the universal joint prosthesis the double threads may be disposed at the same or different pitch or the same or different angle of repose relative to a vertical or horizontal shaft axis. This will influence whether the shaft moves axially at the same rate along the length of the shaft upon insertion. It will also influence the level of compression force in the joint.

The prosthesis can be universally inserted in other joints in the human body such as but not limited to the ankle, thumb, finger knee and hip. It will therefore be recognised by persons skilled in the art that numerous variations and modifications may be made to the invention broadly described herein without departing from the overall spirit and scope of the invention.

1. A universal shaft component capable of fixation in multiple sites in a bone skeleton; wherein the shaft is insertable axially within an internal cavity in bone such that the outer surface of the shaft engages an inner walls of said cavity; wherein the shaft has first and seconds ends wherein one said ends includes a formation which receives and retains a component, the outer surface of said shaft including at least one thread having a pitch geometry which when the shaft is screwed into said bone cavity induces an axial compression force in said bone.

2. A universal shaft component according to claim 1 wherein the axial compression force is evenly distributed in the bone along the length of said at least one thread.

3. A shaft according to claim 2 wherein, the first end includes a flared tapered region and the second end is narrower than the first end.

4. A shaft according to claim 3 wherein the flared tapered regions receives a mating member.

5. A shaft according to claim 4 wherein the shaft has disposed between said first and second ends one helical thread.

6. A shaft according to claim 5 wherein the pitch of the thread varies as the thread travels axially along the shaft.

7. A shaft according to claim 6 wherein the angle of repose of the thread relative to a vertical or horizontal axis varies along the thread as the thread travels axially along the shaft.

8. A shaft component according to claim 7 wherein, the helical thread is continuous along a region approximating a middle third of the shaft.

9. A shaft according to claim 8 wherein the thread undergoes a variation in pitch from a slow thread near the flared end to a fast thread towards the second end.

10. A shaft according to claim 9 wherein the thread has a gradual but regular variation in pitch along the length of the thread from a fast to slow thread.

11. A shaft according to claim 10 wherein an even compression force is induced in the bone due to differences in travel rates induced on insertion of the shaft by the thread.

12. A shaft according to claim 1, further comprising a profile part at the flared end which receives and retains a detachable joining component by male female or female male interfitting.

13. A shaft according to claim 1 capable of insertion in a glenohumeral joint of the shoulder.

14. A shaft according to claim 1 capable of insertion in a distal end of a femur as a partial knee component.

15. A shaft according to claim 1 capable of insertion in a proximal end of a tibia as a partial knee component.

16. A shaft according to claim 1 capable of insertion in a proximal phalange to form a part finger joint component.

17. A shaft according to claim 1 capable of insertion in a distal end of a tibia.

18. A shaft according to claim 1 capable of insertion in a talus.

19. A shaft according to claim 1 capable of insertion in a proximal femur.

20. A universal shaft component capable of fixation as an anchorage in multiple sites in a bone skeleton; wherein the shaft is insertable axially within an internal cavity bone such that the outer surface of the shaft engages an inner wall of said cavity; wherein the shaft has first and seconds ends wherein one said ends includes a formation which receives and retains a joining component, the outer surface of said shaft including first and second spaced apart helical threads; wherein the threads having a pitch geometry which when the shaft is screwed into said bone cavity induces an axial compression force in said bone.

21. A universal shaft component according to claim 20 wherein the axial compression force is evenly distributed in the bone along the length of said at least one thread.

22. A shaft according to claim 21 wherein, the first end includes a flared tapered region and the second end is narrower than the first end.

23. A shaft according to claim 22 wherein each thread has a different pitch.

24. A shaft according to claim 23 wherein the angle of repose of a first of the threads relative to a vertical or horizontal axis of the shaft is different from the angle of repose of the second shaft.

25. A shaft component according to claim 24 wherein, the first thread is disposed in a region of the shaft near the flared tapered region and the second thread is disposed in a region approximating a longitudinal center of the shaft.

26. A shaft according to claim 25 wherein the first thread is a slow thread and the second thread is a fast thread.

27. A shaft according to claim 26 wherein the first thread causes a slower axial travel of the shaft than the second thread upon screwing the shaft into a bone cavity.

28. A shaft according to claim 27 wherein the even compression force is induced in the bone due to differences in travel rates induced on insertion of the shaft by the thread.

29. A shaft according to claim 20 further comprising a profile part at the flared end which receives and retains a detachable joining component.

30. A shaft according to claim 29 wherein the flared end includes a tapered female recess which receives said joining member.

31. A shaft according to claim 29 wherein one of the threads has a gradual but regular variation in pitch along the length of the thread from a fast to slow thread.

32. A shaft according to claim 20 capable of insertion in a glenohumeral joint of a shoulder.

33. A shaft according to claim 20 capable of insertion in a distal end of a femur as a partial knee replacement component.

34. A shaft according to claim 20 capable of insertion in a proximal end of a tibia as a partial knee replacement component.

35. A shaft according to claim 20 capable of insertion in a proximal phalange as a partial finger joint replacement.

36. A shaft according to claim 20 capable of insertion in a distal end of a tibia as a partial ankle joint replacement.

37. A shaft according to claim 20 capable of insertion in a talus.

38. A universal shaft component capable of insertion as an anchorage in skeletal bone including a proximal humerus, phalange, distal or proximal tibia, distal or proximal femur, or thumb wherein the shaft is insertable axially within an internal bone cavity such that the outer surface of the shaft engages inner walls of said cavity, characterised in that the shaft has a priximal end and a distal end and on said outer surface of said shaft between said ends, at least one thread such that when the shaft is screwed into said bone cavity the at least one thread induces an axial compression force in said bone and distributes that compression force evenly along the bone over the length of the at least one thread.

39. A universal shaft component for use as an anchorage in a bone and which is capable of forming at least part of a joint replacement in an ankle, hip, finger, thumb, shoulder or knee; the shaft comprising a threaded outer surface which is profiled to induce a compression force in the bone in which it is inserted to enhance fixation and further comprising a flared end and a narrow end, the flared end having a formation which receives and retains a joining member.
